(19) United States
(12) Patent Application Publication
(10) Pub. No.: US 2018/0360410 A1
(43) Pub. Date: Dec. 20, 2018

(54) SYSTEMS AND METHODS FOR CONTROLLING A MEDICAL IMAGING DEVICE VIA A MOBILE DEVICE

(71) Applicant: SHANGHAI UNITED IMAGING HEALTHCARE CO., LTD., Shanghai (CN)

(72) Inventor: Hongyu SUN, Shanghai (CN)

(73) Assignee: SHANGHAI UNITED IMAGING HEALTHCARE CO., LTD., Shanghai (CN)

(21) Appl. No.: 16/012,881

(22) Filed: Jun. 20, 2018

(30) Foreign Application Priority Data
Jun. 20, 2017 (CN) 201720717234.7
Jun. 28, 2017 (CN) 201720769505.3

(51) Int. Cl. A61B 6/00; A61B 5/055; A61B 6/03; G16H 30/40

(52) U.S. Cl. CPC A61B 6/548; A61B 5/055; A61B 6/563; G06T 11/003; A61B 6/037; G16H 30/40; A61B 6/032

(57) ABSTRACT

A medical imaging system is provided. The medical imaging system may include a mobile device configured to remotely control a medical imaging device. The medical imaging device may include a scanner, a control module, and a second communication port. The second communication port may be configured to receive an instruction from the mobile device via a network. The scanner may scan the object and generate scanning data related to the object in response to a scan instruction received by the second communication port from the mobile device via the network. The control module may control movement of at least one of the gantry of the scanner or the scanning table in response to a control instruction received by the second communication port from the mobile device via the network.

### CROSS-REFERENCE TO RELATED APPLICATIONS

This application claims priority to Chinese Patent Application No. 201720717234. 7, filed on Jun. 20, 2017, and Chinese Patent Application No. 201720769505. 3, filed on Jun. 28, 2017, the entire contents of which are hereby incorporated reference.

### TECHNICAL FIELD

The present disclosure generally relates to a medical imaging device, and more specifically, relates to systems and methods for controlling a medical imaging device via a mobile device.

### BACKGROUND

Medical imaging systems are used for a variety of applications in the field of medicine. For example, medical imaging systems such as computed tomography (CT), magnetic resonance imaging (MRI), positron emission tomography (PET) may be used for diagnosis purposes. Merely by way of example, in a CT system, a user (e.g., a doctor) may control to perform one or more operations (e.g., scanning of an object and/or image reconstruction) via a terminal device. In some embodiments, the terminal device may stay at a fixed location. Besides, the terminal device may communicate with other components of the CT system via a wired network. Thus, the user (e.g., the doctor) may have to control the scan of the object or image reconstruction at a fixed location, and such a setup cannot make a quick response during the scan process. It is desirable to utilize a mobile device that wirelessly communicates with other components of the medical system to control these components.

### SUMMARY

In one aspect of the present disclosure, a medical imaging device is provided. The medical imaging device may include at least one processor, at least one storage, and a communication platform connected to a network. The medical imaging device may include a scanner, a scanning table, a communication port, and a control module. The scanner may be configured to generate scanning data related to an object. The scanner may include a gantry, a radiation source, and a detector. The radiation source may be mounted to the gantry and configured to emit radiation to the object. The detector may be mounted to the gantry opposite to the radiation source and configured to detect radiations emitted from the radiation source. The scanning table may be configured to move with respect to the gantry of the scanner. The control module may be configured to control a movement of at least one of the gantry of the scanner or the scanning table. The communication port may be configured to receive an instruction from a mobile device via the network. The scanner may scanner the object and generate the scanning data related to the object in response to a scan instruction received by the communication port from the mobile device via the network. The control module may control the movement of at least one of the gantry or the scanning table in response to a control instruction received by the communication port from the mobile device via the network.

In some embodiments, to control the movement of the gantry of the scanner, the control module may be further configured to perform at least one of adjusting a tilt angle of the gantry relative to a horizontal plane, controlling a deflection angle of the gantry, controlling a rotation velocity of the gantry, or controlling a rotation direction of the gantry.

In some embodiments, to control the movement of the scanning table, the control module may be further configured to perform at least one of controlling a moving distance of the scanning table with respect to the gantry, controlling a moving direction of the scanning table with respect to the gantry, controlling a moving velocity of the scanning table, controlling a moving acceleration of the scanning table, or controlling a tilt angle of the scanning table.

In some embodiments, the medical imaging device may further include an image reconstruction module. The image reconstruction module may be configured to reconstruct one or more images of the object based on the scanning data related to the object in response to a data processing instruction received by the communication port from the mobile device via the network.

In some embodiments, the communication port may be further configured to transmit at least one of the scanning data related to the object or the one or more images of the object via the network to be displayed on the mobile device.

In some embodiments, the gantry of the scanner may be configured with a Universal Serial Bus (USB) port or a charging connector to charge the mobile device.

In another aspect of the present disclosure, a medical imaging method is provided. The medical imaging method may be implemented on one machine including at least one processor, at least one storage, and a communication platform connected to a network. The method may include receiving a scan instruction from a mobile device via the network, and generating scanning data related to an object by scanning the object in response to the scan instruction. The method may also include receiving a control instruction from a mobile device via the network and controlling the movement of at least one of a gantry of a scanner or a scanning table in response to the control instruction.

In yet another aspect of the present disclosure, a medical imaging device is provided. The mobile device may include a mobile device and a medical imaging device. The mobile device may be configured to remotely control the medical imaging device. The medical imaging device may include a scanner, a control module, and a second communication port. The second communication port may be configured to receive an instruction from the mobile device via a network. The scanner may be configured to scan an object and generate scanning data related to the object in response to a scan instruction received by the second communication port from the mobile device via the network. The control module may be configured to control a movement of at least one of the gantry of the scanner or the scanning table in response to a control instruction received by the second communication port from the mobile device via the network.

In some embodiments, to control the movement of the gantry of the scanner, the control module may be further configured to perform at least one of adjusting a tilt angle of the gantry relative to a horizontal plane, controlling a deflection angle of the gantry, controlling a rotation velocity of the gantry, or controlling a rotation direction of the gantry.

In some embodiments, to control the movement of the scanning table, the control module may be further configured to perform at least one of controlling a moving distance of the scanning table with respect to the gantry, controlling a moving direction of the scanning table with respect to the gantry, controlling a moving velocity of the scanning table, controlling a moving acceleration of the scanning table, or controlling a tilt angle of the scanning table.

In some embodiments, the medical imaging system may further include an image reconstruction module. The image reconstruction module may be configured to reconstruct one or more images of the object based on the scanning data related to the object in response to a data processing instruction received by the second communication port from the mobile device via the network.

In some embodiments, the second communication port may be further configured to transmit at least one of the scanning data related to the object or the one or more images of the object via the network to be displayed on the mobile device.

In some embodiments, the mobile device may include at least one processor, a user interface, a display, a first communication port. The first communication port may be connected to the network and configured to enable communications between the mobile device and the medical imaging device. The at least one processor may be configured to generate an instruction based on one or more user inputs. The first communication port may be configured to transmit the instruction to the medical imaging device, which may cause the medical imaging device to operate in response to the instruction.

In some embodiments, the mobile device may further include a camera.

In some embodiments, the mobile device may further include security device configured to lock the mobile device.

In some embodiments, the scanner may include at least one of a Universal Serial Bus (USB) port or a charging connector to charge the mobile device.

In some embodiments, the scanner may further include an outer housing and an inspection chamber. The outer housing may include a load-bearing component. The load-bearing component may be used to accommodate the mobile device. The USB port or the charging connector may be located around or inside the load-bearing component. The inspection chamber may be surrounded by the outer housing and extended along a longitudinal direction. The inspection chamber may be used to accommodate the scanning table.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical imaging system according to some embodiments of the present disclosure;

FIG. 2 is a schematic diagram illustrating a mobile device and a scanning device according to some embodiments of the present disclosure;

FIG. 3 is a schematic diagram illustrating an exemplary remote control system according to some embodiments of the present disclosure;

FIG. 4 is a schematic diagram illustrating an exemplary medical imaging system according to some embodiments of the present disclosure;

FIG. 5 is a schematic diagram illustrating an exemplary medical imaging system according to some embodiments of the present disclosure;

FIG. 6 is a flowchart illustrating an exemplary process for controlling the movement of a gantry and/or a scanning table according to some embodiments of the present disclosure;

FIG. 7A is a block diagram illustrating an exemplary mobile device according to some embodiments of the present disclosure;

FIG. 7B is a block diagram illustrating an exemplary medical imaging device according to some embodiments of the present disclosure;

FIG. 8 is a schematic diagram illustrating an exemplary mobile device according to some embodiments of the present disclosure;

FIG. 9 is a flowchart illustrating an exemplary process for controlling movement of a gantry or a scanning table according to some embodiments of the present disclosure;

FIG. 10 is a flowchart illustrating an exemplary process for scanning an object according to some embodiments of the present disclosure; and

FIG. 11 is a flowchart illustrating an exemplary process for reconstructing one or more images of an object according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “unit,” “module,” and/or “block” used herein are one method to distinguish different components, elements, parts, section or assembly of different level in ascending order. However, the terms may be displaced by other expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included of connected logic components, such as gates and flip-flops, and/or can be included of programmable units, such as programmable gate arrays or processors. The modules/units/blocks or computing device functionality described herein may be implemented as software modules/units/blocks but may be represented in hardware or firmware. In general, the modules/units/blocks described herein refer to logical modules/units/blocks that may be combined with other modules/units/blocks or divided into sub-modules/sub-units/sub-blocks despite their physical organization or storage.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

An aspect of the present disclosure relates to systems and methods for controlling a medical imaging device via a mobile device. The medical imaging device and the mobile device may be parts of a medical imaging system. In some embodiments, the mobile device may communicate with the medical imaging device via a network. Merely by way of example, the medical imaging device may receive a control instruction from the mobile device via the network. The medical imaging device may control the movement of at least one of a gantry or a scanning table in response to the control instruction. As another example, the medical imaging device may receive a scan instruction from the mobile device via the network, and the medical imaging device may scan an object and generate scanning data related to the object in response to the scan instruction.

The following description is provided to help better understand the controlling of the medical imaging device by the mobile device. This is not intended to limit the scope the present disclosure. For persons having ordinary skills in the art, a certain amount of variations, changes, and/or modifications may be deducted under the guidance of the present disclosure. Those variations, changes, and/or modifications do not depart from the scope of the present disclosure.

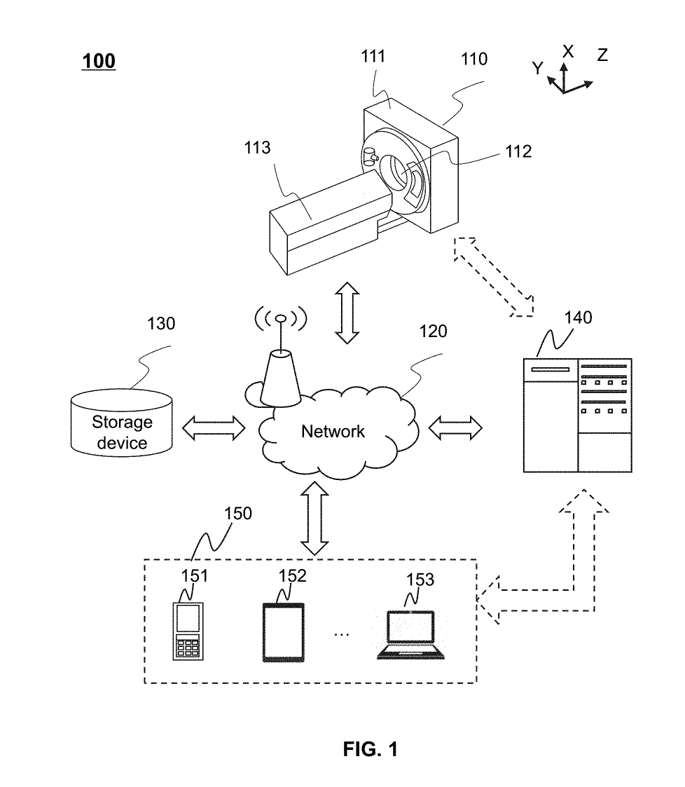

FIG. 1 is a schematic diagram illustrating an exemplary medical imaging system 100 according to some embodiments of the present disclosure. For illustration purposes, the medical imaging system may also be referred to as imaging system. The medical imaging system (or the imaging system) 100 may include a scanning device 110, a network 120, a storage device 130, a processing device 140, and a mobile device 150. The components in the medical imaging system 100 may be connected in one or more of various ways. For example, the scanning device 110 may be connected to the processing device 140 through the network 120. As another example, the scanning device 110 may be connected to the processing device 140 directly as indicated by the bi-directional arrow in dotted lines linking the scanning device 110 and the processing device 140.

The scanning device 110 (also referred to as a scanner) may scan an object and generate scanning data and image(s) related to the object. The object may be a biological object (e.g., a patient, an animal) or a non-biological object (e.g., a man-made object). For example, the object may include an organ, a body, an object, an injured part, a tumor, or the like, or any combination thereof. In the present disclosure, “object” and “subject” are used interchangeably. The scanning device 110 may include but is not limited to a computed tomography (CT) device, a computed tomography angiography (CTA) device, a positron emission tomography (PET) device, a single photon emission tomography (SPECT) device, a magnetic resonance imaging (MRI) device, a digital subtraction angiography (DSA) device, an ultrasonic scanning (US) device, a thermal tomography (TTM) device, etc. In some embodiments, the scanning device may be solely used. Alternatively or additionally, the scanning device may be used with a combination (e.g., a PET-CT system).

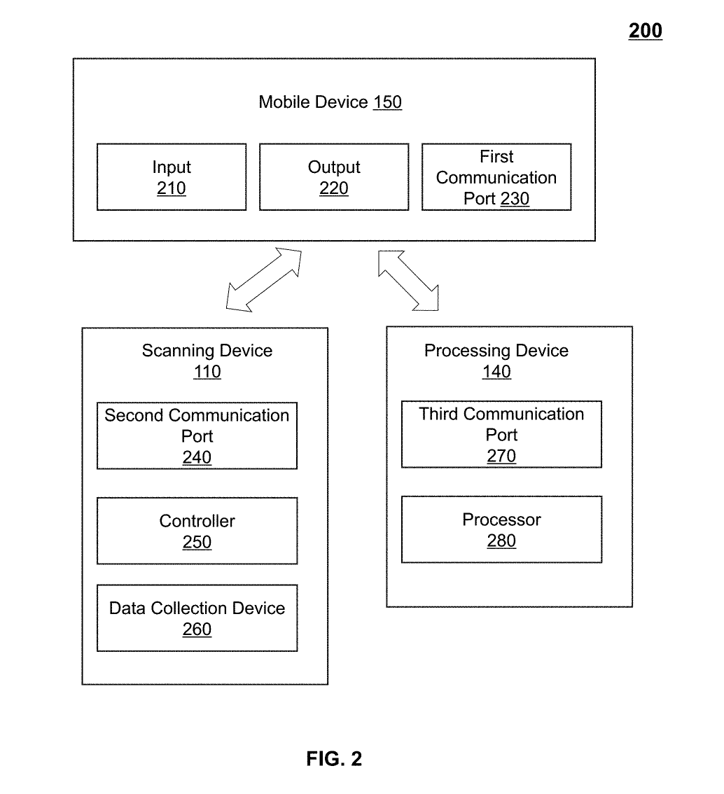

In some embodiments, the scanning device 110 may include a gantry 111 and a scanning table 113. The gantry 111 may be configured to support one or more components of the scanning device 110 (e.g., a detector, a radiation source). The scanning table 113 may be configured to support the object. The gantry 111 may include an inspection chamber 112 surrounded by an outer housing. The inspection chamber 112 may extend along a longitudinal direction (i.e., Z-axis direction as illustrated in FIG. 1). The inspection chamber 112 may be configured to accommodate the scanning table 113. In some embodiments, the outer housing may include a front end near the scanning table 113 and a rear end away from the scanning table 113. The front end may include a load-bearing component, for example, a groove, a shelf, a fastening structure, or the like. The load-bearing component may fix the mobile device 150 at the front end vertically. For example, the outer housing of the gantry 111 may include a groove in which the mobile device 150 is placed vertically. As another example, the outer housing of the gantry 111 may include a shelf on which the mobile device 150 is placed vertically. In some embodiments, a USB port or a charging connector may be located around or inside the load-bearing component. The USB port or the charging connector may charge the mobile device 150 via a wired or wireless connection. Detailed descriptions of the USB port or the charging connector be found elsewhere in the present description (e.g., FIG. 2 and the descriptions thereof).

The scanning device 110 may further include a detector and a radiation source (not shown in FIG. 1). The detector and the radiation source may be oppositely mounted to the gantry 111. The radiation source may emit radiation rays to scan an object that is placed on the scanning table 113. The radiation rays may include X-rays, y-rays, a-rays, ultraviolet, laser, neutron, proton, or the like, or any combination thereof. The detector may detect radiations (e.g., X-rays) emitted from the radiation source (e.g., a CT system). In some embodiments, the detector may include a plurality of detector units. The detector units may include a scintillation detector (e.g., a cesium iodide detector) or a gas detector. The detector units may be arranged in a single row or multiple rows. In some embodiments, the radiation source may be omitted in some medical imaging systems, for example, in a positron emission tomography (PET) system. In the PET system, the object may be injected with a radioactive substance (e.g., radiopharmaceutical). The detector may detect radiations (e.g., gamma photons) emitted from the radioactive substance.

The network 120 may facilitate exchange of information and/or data. In some embodiments, one or more components in the medical imaging system 100 (e.g., the scanning device 110, the storage device 130, the processing device 140, or the mobile device 150) may send information and/or data to other component(s) in the medical imaging system 100 via the network 120. For example, the processing device 140 may obtain scanning data from the scanning device 110 via the network 120. As another example, the processing device 140 may obtain user instructions from the mobile device 150 via the network 120. In some embodiments, the network 120 may be any type of wired or wireless network, or combination thereof. Merely by way of example, the network 120 may include a cable network, a wireline network, an optical fiber network, a telecommunications network, an intranet, an Internet, a local area network (LAN), a wide area network (WAN), a wireless local area network (WLAN), a metropolitan area network (MAN), a wide area network (WAN), a public telephone switched network (PSTN), a BluetoothTM network, a ZigBee™ network, a near field communication (NFC) network, a cellular network (e.g., GSM, CDMA, 3G, 4G), or the like, or any combination thereof. In some embodiments, the network 120 may include one or more network access points. For example, the network 120 may include wired or wireless network access points such as base stations and/or internet exchange points through which one or more components of the medical imaging system 100 may be connected to the network 120 to exchange data and/or information.

The storage device 130 may store data and/or instructions. In some embodiments, the storage device 130 may store data obtained from the processing device 140 and/or the mobile device 150. In some embodiments, the storage device 130 may store data and/or instructions that the processing device 140 may execute or use to perform exemplary methods described in the present disclosure. In some embodiments, the storage device 130 may store data and/or information received by the mobile device 150, for example, the input received through a user interface of the mobile device 150. In some embodiments, the storage device 130 may store data and/or information generated during an imaging process, for example, scanning data, one or more generated images. In some embodiments, the storage device 130 may store data related to an object. The data related to the object may include basic information of the object, medical information of the object, etc.

In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 120 to communicate with one or more components in the medical imaging system 100 (e.g., the processing device 140, the mobile device 150). One or more components in the medical imaging system 100 may access the data or instructions stored in the storage device 130 via the network 120. In some embodiments, the storage device 130 may be directly connected to or communicate with one or more components in the medical imaging system 100 (e.g., the processing device 140, the mobile device 150). In some embodiments, the storage device 130 may be part of the processing device 140.

The processing device 140 may process data obtained from the scanning device 110, the storage device 130, or the mobile device 150. The processing of the data may include data pre-processing, data converting processing, data cleaning processing, data fitting processing, or the like, or any combination thereof. The data may include data related to an object, image data, file data, training data, or the like, or any combination thereof. In some embodiments, the processing device 140 may process the data based on a data processing instruction received from the mobile device 150. For example, the processing device 140 may manage and/or process data related to the object, including addition, deletion, ranking, screening, analyzing, registration, etc., based on a data processing instruction received from the mobile device 150. In some embodiments, the processing device may receive the data processing instruction from the mobile device 150 via the network 120.

The processing device 140 may be a central processing unit (CPU), a digital signal processor (DSP), a system on a chip (SoC), a microcontroller unit (MCU), or the like, or any combination thereof. In some embodiments, the processing device 140 may be a single server or a server group. The server group may be centralized or distributed. In some embodiments, the processing device 140 may be local or remote. For example, the processing device 140 may access information and/or data stored in the scanning device 110, or the storage device 130, via the network 120. As another example, the processing device 140 may be directly connected to the scanning device 110, or the storage device 130, to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The mobile device 150 may receive and/or transmit data or signal(s). The data or signal(s) may be inputted by a user, or obtained from other components of the medical imaging system 100. For example, the mobile device 150 may receive data related to the object, scanning parameters, reconstruction parameters inputted by the user. As another example, the mobile device 150 may receive a data processing result (e.g., one or more images) transmitted by the processing device 140. As a further example, the mobile device 150 may receive signal(s) inputted by the user. The inputted signal(s) may include a scan instruction, a data management instruction (e.g., a data processing instruction), a control instruction, or the like, or any combination thereof.

Alternatively or additionally, the mobile device 150 may further output data. For example, the mobile device 150 may display the data related to the object inputted by the user. As another example, the mobile device 150 may display the data processing result transmitted by the processing device 140. The mobile device 150 may be any device having input and output functions, such as a mobile device 151, a tablet computer 152, a laptop computer 153, or the like, or any combination thereof. In some embodiments, the mobile device 150 may be a tablet computer (e.g., an iPad™)

In some embodiments, the mobile device 151 may include a smart home device, a wearable device, a smart mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a smart bracelet, a smart footgear, smart glasses, a smart helmet, a smartwatch, smart clothing, a smart backpack, a smart accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google Glass, an Oculus Rift, a Hololens, a Gear VR, etc.

In some embodiments, the mobile device 150 may process data and/or information entered by the user. In some embodiments, the data and/or information may be entered into the mobile device 150 via a user interface, including a keyboard, a mouse, a bottom, a screen, a microphone, or the like, or any combination thereof. The data and/or information may include data relating to an object, one or more scanning parameters, one or more reconstruction parameters, or the like, or any combination thereof. In some embodiments, the mobile device 150 may process the data and/or information to generate an instruction (e.g., a control instruction, a scan instruction, a data processing instruction).

One or more components of the medical imaging system 100 may communicate with each other via a wired or wireless connection. For example, components of the medical imaging system 100 may communicate data, signal(s), and/or information with each other via the wired or wireless connection. In some embodiments, the mobile device 150 may communicate with the storage device 130, the scanning device 110, and/or the processing device 140 via the wireless connection. For example, the mobile device 150 may receive a data processing result (e.g., one or more images) of the processing device 140. As another example, the mobile device 150 may transmit a scan instruction and/or a control instruction to the scanning device 110 to control the operation of the scanning device 110.

FIG. 2 is a schematic diagram 200 illustrating a mobile device and a scanning device according to some embodiments of the present disclosure. The mobile device 150 may include an input 210, an output 220, and a first communication port 230. The scanning device 110 may include a second communication port 240, a controller 250, and a data collection device 260. The processing device 140 may include a third communication port 270 and a processor 280.

The input 210 may be also referred to as a user interface, which may support data and/or information interactions between the mobile device 150 and a user. For example, the input 210 may receive data or signal(s) inputted by a user. The data may include data related to an object (e.g., a patient), scanning parameters, reconstruction parameters, or the like, or any combination thereof. The data related to the object may include basic information of the object, disease condition information (also referred to as medical information), medicine allergy information, diagnostic information, or the like, or any combination thereof. The basic information may include name, gender, age, profession, native place, workplace, home address, or the like, or any combination thereof. The disease condition information may include current disease condition, medical history, family medical history, or the like, or any combination thereof. The diagnostic information may include diagnostic result, therapeutic regimen, assay result, or the like, or any combination thereof. The scanning parameters may include scanning time, irradiation dose, slice thickness of scanning, scanning angles, tube voltage, tube current, tilt angle of a gantry, height of the gantry, or the like, or any combination thereof. The reconstruction parameters may include a reconstruction function, a size of a reconstruction image, iteration times of reconstruction, a size of a reconstruction matrix, slice thickness of reconstruction, or the like, or any combination thereof.

The signal(s) (or information) inputted by the user may include a text signal, an audio signal, a video signal, an image signal, a graphic signal, or the like, or any combination thereof. The signal(s) (or information) inputted by the user may include a scan instruction, a data management instruction (also referred to as a data processing instruction), a control instruction, or the like, or any combination thereof. The scan instruction may be used to control the scanning device 110 to scan an object (e.g., a patient). The data processing instruction may be used for controlling, processing and/or analyzing data. The data may include data related to the object (e.g., a patient), image data, film data, training data, or the like, or any combination thereof. For example, the data processing instruction may be used to manage and/or process the data related to the object, including addition, deletion, ranking, screening, analyzing, registration, etc. As another example, the data processing instruction may be used to manage and/or process image data, including image reconstruction, image segmentation, image amplification, image reduction, image analysis, etc.

The control instruction may be used to control one or more components of the medical imaging system 100. In some embodiments, the control instruction may control the operation of the scanning device 110. For example, the control instruction may control the position of the scanning table (e.g., the scanning table 113) and/or the gantry (e.g., the gantry 111), control a scan process, control the setting of scanning parameter(s), etc. In some embodiments, the control instruction may control the operation of a printing device (not shown in FIG. 2). For example, the control instruction may control the printing device to print image(s) and/or film(s). In some embodiments, the control instruction may control the processing device 140 to process and/or analyze image data. The image data may include a two-dimensional image, two-dimensional image data, a three dimensional image, three-dimensional image data, a four dimensional image, four-dimensional image data, or the like, or any combination thereof. For example, the control instruction may control the processing device 140 to perform an image reconstruction.

In some embodiments, the input 210 may include a character input device (e.g., a keyboard), an optical reading device (e.g., an optical mark reader, an optical character reader, etc.), a graphic input device (e.g., a mouse, a joystick, a light pen), an image input device (e.g., a camera, a scanner, a fax machine), an analog input device (e.g., a language analog-digital conversion recognition system), or the like, or any combination thereof.

In some embodiments, the input 210 may include a camera. The camera may be used to take a photo, scan an identification code, etc. The identification code may include a quick response (QR) code, a bar code, etc. For example, before a scan process, a user (e.g., a doctor) may take a photo of an object (or a patient) using the camera to record data related to the object. As another example, before a scan process, a user (e.g., a doctor) may scan a clinical case of an object (e.g., a patient), and/or a QR code or a bar code on wristbands of the object (e.g., the patient). The mobile device 150 may retrieve data related to the object (e.g., the patient) based on the result of the camera scan.

The output 220 may be used to display data. The data may include data related to an object (e.g., a patient), image data, film data, training data, or the like, or any combination thereof. The data may be displayed in various ways, including sound (e.g., voice), visual (e.g., text, video, graphic, etc.), or the like, or any combination thereof. In some embodiments, the output 220 may include a display, a loudspeaker, an amplifier, or the like, or any combination thereof. In some embodiments, the output 220 may include a display. The display may display an input interface. A user may input data or signal(s) via the input interface.

The first communication port 230 may be configured to establish a connection between the mobile device 150 and other components of the medical imaging system 100, and enable data and/or signal transmission between the mobile device 150 and other components of the medical imaging system 100. For example, the first communication port 230 may establish a wireless connection between the mobile device 150, the scanning device 110, and/or the processing device 140. The wireless connection may include, for example, a Bluetooth connection, a wireless network connection, a WLAN link, a ZigBee connection, a mobile network connection (e.g., 3G, 4G, 5G network, etc.), or the like, or any combination thereof.

The first communication port 230 may transmit data and/or signal(s) inputted by a user to other components of the medical imaging system 100. For example, the first communication port 230 may transmit data related to the object, scanning parameters, reconstruction parameters inputted by the user to the storage device 130 for storage. As another example, the first communication port 230 may transmit a control instruction inputted by the user to the scanning device 110 to control the operation of the scanning device 110. In some embodiments, the first communication port 230 may also process the signal(s) inputted by the user and then transmit the processed signal(s) to other components of the medical imaging system 100. For example, the first communication port 230 may convert a text signal, an audio signal, a video signal, etc. into an electrical signal (e.g., a digital signal, an analog signal), and then transmit the converted signal to other components of the medical imaging system 100.

The first communication port 230 may receive data and/or signal(s) from the scanning device 110 and/or the processing device 140. For example, the first communication port 230 may receive a data processing result transmitted by the processing device 140. The data processing result may include a processing result of data related to the object, a processing result of image data of the object, or the like, or any combination thereof. In some embodiments, the first communication port 230 may receive a reconstruction image transmitted by the processing device 140. Furthermore, the output 220 may display the received reconstruction image.

In some embodiments, the mobile device 150 may also include a security device. The security device may be used to lock the mobile device 150. The security device may include a screen locker, a password locker, or the like, or any combination thereof. For example, the security device may be a screen locker. A user may close or lock the screen using the screen locker to avoid misoperation or other's use. As another example, the security device may be a gesture password, a digital password, a fingerprint password, etc. The security device may lock the mobile device 150 when the user not input a password or input an incorrect password.

In some embodiments, the mobile device 150 may also include a storage device (not shown in FIG. 2). The storage device may store data and/or signal(s), for example, the data and/or signal(s) inputted by the user. In some embodiments, the mobile device 150 may also include a processor (not shown in FIG. 2). The processor may be used to process data or image(s). In some embodiments, the processor may reconstruct one or more images of an object based on scanning data collected by the scanning device 110.

The second communication port 240 of the scanning device 110 may be configured to establish a connection between the scanning device 110 and other components of the medical imaging system 100, and enable data and/or signal transmission between the scanning device 110 and other components of the medical imaging system 100. For example, the second communication port 240 may receive scanning parameter(s), a scan instruction, a control instruction, etc. inputted by a user via the mobile device 150. As another example, the second communication port 240 may transmit scanning data generated by the scanning device 110 to the mobile device 150 and/or the processing device 140. The second communication port 240 may be similar to the first communication port 230, and the descriptions thereof are not repeated herein.

The controller 250 (also referred to as a control module) may control the operation of the scanning device 110. In some embodiments, the controller 250 may control the operation of the scanning device 110 based on signal(s) received by the second communication port 240. In some embodiments, the controller 250 may control the operation of the scanning device 110 based on the scanning parameters, the scan instruction, the control instruction inputted by the user via the mobile device 150. For example, the controller 250 may set the scanning parameters of the scanning device 110 based on the scanning parameters inputted by the user via the mobile device 150.

The data collection device 260 may be configured to collect scanning data. The data collection device 260 may include a radiation source (e.g., the radiation source described in FIG. 1) and a detector (e.g., the detector described in FIG. 1). The radiation source may emit radiation rays to an object. The radiation rays may include X-rays, gamma rays, ultraviolet rays, laser, neutron, proton, alpha rays, or the like, or any combination thereof. The detector may be configured to receive the radiation rays passed through the object.

In some embodiments, the scanning device 110 may include a USB port or a charging connector. The USB port or a charging connector may charge the mobile device 150 via a wired or wireless connection. For example, the scanning device 110 may include a USB port, and the mobile device 150 may have a charging hole (e.g., another USB port) matching with the USB port. The USB port and the charging hole may be connected through a USB cable, which may facilitate to charge the mobile device. As another example, the scanning device 110 may include a charging connector (also referred to as a socket), and the mobile device 150 may include a charging plug. The charging plug may be inserted into the charging connector to charge the mobile device 150. In some embodiments, the USB port may be the second communication port 240. In some embodiments, the USB port or the charging connector may be located around or inside a load-bearing component of the scanning device 110. Detailed descriptions of the load-bearing component may be found elsewhere in the present disclosure (e.g., FIG. 1 and the descriptions thereof).

The third communication port 270 of the processing device 140 may be configured to establish a connection between the processing device 140 and other components of the medical imaging system 100, and enable data and/or signal transmission between the processing device140 and other components of the medical imaging system 100. For example, the third communication port 270 may establish a connection between the processing device 140 and the mobile device 150, and receive data and/or signal(s) transmitted by the first communication port 230 of the mobile device 150. The data and/or signal(s) may include reconstruction parameters, a data processing instruction, etc. inputted by the user. As another example, the third communication port 270 may transmit a data processing result to the mobile device 150. The third communication port 270 may be similar to the first communication port 230, and the descriptions thereof are not repeated herein.

The processor 280 may be configured to process data and/or image(s). For example, the processor 280 may process data and/or image(s) based on data and/or signal(s) received by the third communication port 270. In some embodiments, the processor 280 may perform image data processing (e.g., image reconstruction) based on reconstruction parameter(s) and/or a data processing instruction inputted by a user. In some embodiments, the processor 280 may determine a scan plan (e.g., determining scanning parameters) based on the data and/or signal(s) inputted by the user. In some embodiments, the processor 280 may include or be an image reconstruction module.

It should be noted that the above descriptions of the mobile device 150, the scanning device 110, and/or the processing device 140 are merely provided for illustration purposes, and are not intended to limit the protection scope of the present disclosure. In some embodiments, one component may be split into a plurality of components. In some embodiments, a plurality of components may be integrated into one component. For persons having ordinary skills in the art, multiple variations and modifications may be made under the teachings of the present disclosure. However, those variations and modifications do not depart from the scope of the present disclosure.

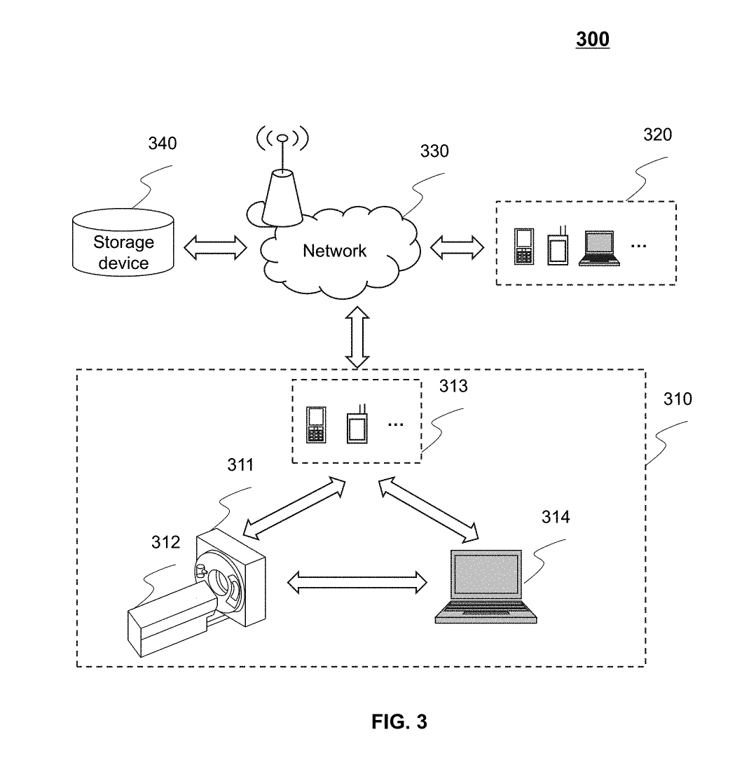

FIG. 3 is a schematic diagram illustrating an exemplary remote control system 300 according to some embodiments of the present disclosure. The remote control system 300 may include a medical imaging system 310, one or more remote control terminals 320, a network 330, and a storage device 340.

The medical imaging system 310 may be configured to perform a scan for an object (e.g., a diagnostic scan for a patient). The medical imaging system 310 may include a scanning device 311, a scanning table 312, one or more mobile devices 313, and a host computer 314 (also referred to as a processing device). The medical imaging system 310 may include a CT device, a MRI device, or the like, or any combination thereof. In some embodiments, the medical imaging system 310 may include a CT device.

In some embodiments, the scanning device 311 and/or the scanning table 312 may be connected to and/or communicate with the mobile device(s) 313 via a signal receiver. The scanning device 311 and/or the scanning table 312 may be connected to and/or communicate with the mobile device 313(s) via a wireless connection, for example, a Bluetooth connection, a WLAN connection, a Wi-Fi connection, or the like, or any combination thereof.

In some embodiments, the mobile device(s) 313 may be configured to control the movement of the scanning device 311 and/or the scanning table 312. The mobile device(s) 313 may control the movement of the scanning device 311 and/or the scanning table 312 based on an input signal inputted by a user, a control signal transmitted by the host computer 314, and/or a control signal received from the network 330. The mobile device(s) 313 may include a computer, a mobile device, a wearable device, or any device that has signal transmission/reception functions. For example, the mobile device(s) 313 may be a remote controller with a function of transmitting and receiving signals. The mobile device(s) 313 may be connected to and/or communicate with the scanning device 311, the scanning table 312, the host computer 314, and/or the network 330 via a signal transmitter and/or an input (also referred to as a signal receiver) (not shown in FIG. 3). The mobile device (s) 313 and the host computer 314 may be connected via a wireless connection, for example, a Bluetooth connection, a WLAN connection, a Wi-Fi connection, or the like, or any combination thereof. The mobile device(s) 313 and the network 330 may be connected via a wireless connection, for example, a Bluetooth connection, a WLAN connection, a Wi-Fi connection, or the like, or any combination thereof. Alternatively, the mobile device(s) 313 and the network 330 may be connected via a wired connection, for example, a metal wire, an optical fiber, or the like, or any combination thereof. The mobile device(s) 313 may be fixed on the scanning device 311 by a load-bearing component of the scanning device 311, for example, a groove, a fastening structure, or the like. Alternatively, the mobile device(s) 313 may be separated from the scanning device 311.

In some embodiments, the host computer 314 (also referred to herein as a processing device) may be configured to receive an input signal inputted by a user and/or a control signal received from the network 330. The host computer 314 may also be configured to transmit the input signal and/or the control signal to the mobile device(s) 313 to control the movement of the scanning device 311 and/or the scanning table 312. The host computer 314 may include a desktop computer, a laptop computer, a server, a processor, or any other device having data processing functions. For example, the host computer 314 may be a desktop computer on which a user can input a signal. The signal may include a text signal, an audio signal, a video signal, an image signal, or the like, or any combination thereof. The host computer 314 may process the signal inputted by the user to obtain a digital signal and/or an analog signal. The host computer 314 may transmit the control signal, which may also be referred to as a scan parameter, to the mobile device(s) 313. The host computer 314 and the mobile device(s) 313 may be connected via a wireless connection, for example, a Bluetooth connection, a WLAN connection, a Wi-Fi connection, or the like, or any combination thereof.

In some embodiments, the host computer 314 may also receive a control signal from the network 330 and transmit the control signal to the mobile device(s) 313. The host computer 314 and the network 330 may be connected via a wireless connection, for example, a Bluetooth connection, a WLAN connection, a Wi-Fi connection, or the like, or any combination thereof. The host computer 314 and the network 330 may be connected via a wired connection, for example, a metal wire, an optical fiber, or the like, or any combination thereof.

In some embodiments, the host computer 314 may receive scanning data collected by the scanning device 311. The scanning device 311 may acquire the scanning data of the object during a scan process. The scanning device 311 may collect scanning data and transmit the scanning data to the host computer 314. The host computer 314 and the scanning device 311 may be connected via a wireless connection, for example, a Bluetooth connection, a WLAN connection, a Wi-Fi connection, or the like, or any combination thereof. The host computer 314 and scanning device 311 may be connected via a wired connection, for example, a metal wire, an optical fiber, or the like, or any combination thereof.

In some embodiments, the remote control terminal(s) 320 may be configured to receive the input signal inputted by the user and transmit the input signal to the network 330. The remote control terminal(s) 320 may include a computer, a mobile device, a wearable device, or any device that has signal transmission/reception functions. The remote control terminal(s) 320 may be a device with a button, a touchscreen device, or any device that has an input. For example, the remote control terminal(s) 320 may be a mobile device with a touchscreen. The remote control terminal(s) 320 may be connected to and/or communicate with the network 330 via a signal transmitter (not shown in FIG. 3). The remote control terminal(s) 320 and the network 330 may be connected via a wireless connection, for example, a Bluetooth connection, a WLAN connection, a Wi-Fi connection, or the like, or any combination thereof. The remote control terminal(s) 320 and the network 330 may be connected via a wired connection, for example, a metal wire, an optical fiber, or the like, or any combination thereof. In some embodiments, the remote control terminal(s) 320 may be omitted.

In some embodiments, the network 330 may be configured to connect the medical imaging system 310, the remote control terminal(s) 320, and/or the storage device 340, and enable data transmission between thereof. The network 330 may be a single network or a combination of multiple networks. For example, the network 330 may include a local area network (LAN), a wide area network (WAN), a public network, a private network, a wireless local area network (WLAN), a virtual network, a metropolitan area network, a public telephone switched network (PSTN), or the like, or any combination thereof. The network 330 may include one or more network access points. For example, the network 330 may include wired or wireless network access points, a base station, or network switching points. A data source may be connected to the network 330 through the access points, and information may be transmitted via the network 330. In some embodiments, the network 330 may be part of the medical imaging system 100.

In some embodiments, the storage device 340 may be configured to store data from the medical imaging system 310 and/or the remote control terminal(s) 320. The data may include data related to the object, including a serial number of the object, movement of the scanning table 312 and/or the gantry 410 during scanning the object, scanning data collected by the medical imaging system 310. The storage device 340 may refer to a device having a storage function, for example, various memories, Random Access Memory (RAM), Read Only Memory (ROM), or the like, or any combination thereof. The storage device 340 may be local or remote. The storage device 340 and the network 330 may be connected via a wireless connection, for example, a Bluetooth connection, a WLAN connection, a Wi-Fi connection, or the like, or any combination thereof. The storage device 340 and the network 330 may be connected via a wired connection, for example, a metal wire, an optical fiber, or the like, or any combination thereof. In some embodiments, the storage device 340 may be part of the medical imaging system 310.

In some embodiments, the remote control terminal(s) 320 may process the input signal received from the user and transmit the processed signal to the network 330. The input signal may include a text signal, an audio signal, a video signal, an image signal, a graphic signal, or the like, or any combination thereof. The remote control terminal(s) 320 may analyze the received input signal and determine information related to one or more components of the medical imaging system 310. The remote control terminal(s) 320 may also convert the received input signal to a control signal (e.g., a digital control signal and/or an analog control signal), and transmit the control signal to the network 330. The network 330 may transmit the control signal to the medical imaging system 310 and/or the storage device 340. The storage device 340 may store the control signal (e.g., the digital control signal, the analog control signal). The medical imaging system 310 may control the movement of one or more components of the medical imaging system 310 based on the control signal (e.g., the digital control signal, the analog control signal). In some embodiments, the medical imaging system 310 may control the movement of the scanning table 312 and/or the scanning device 311 based on the control signal.

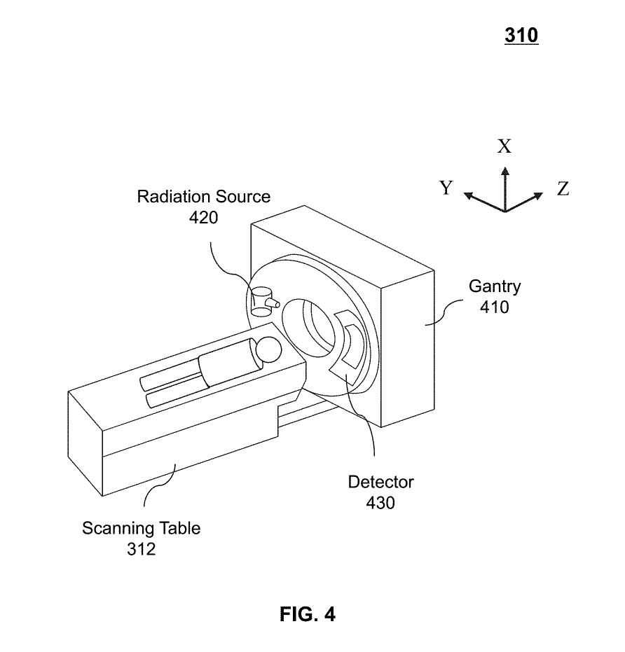

FIG. 4 is a schematic diagram illustrating an exemplary medical imaging system 310 according to some embodiments of the present disclosure. The medical imaging system 310 may include a scanning device 311 and a scanning table 312. The scanning device 311 may include a gantry 410, a radiation source 420, and a detector 430. The gantry 410 may be configured to support one or more components of the scanning device 311 (e.g., the radiation source 420, the detector 430). In some embodiments, the gantry 410 may include an inspection chamber surrounded by an outer housing. The inspection chamber may have a circular shape. The inspection chamber may extend along a longitudinal direction, i.e., the Z-axis direction as illustrated in FIG. 4. The outer housing of gantry 410 may have a load-bearing component such as a groove, a fastening structure, or the like. The load-bearing component may be configured to accommodate the mobile device 313. The size of the fixed device may match with the size of the mobile device 313. The radiation source 420 may be configured to emit radiation rays or signals to the object. The radiation rays may include X-rays, gamma rays, or the like, or any combination thereof. The detector 430 may be configured to receive radiation rays passed through the object. The detector 430 and the radiation source 420 may be oppositely mounted to the gantry 410. The scanning table 312 may be configured to place the object. The object may include a human, an object, or the like, or any combination thereof. In some embodiments, in the medical imaging system 310, the object may lie on the scanning table 312 along the Z-axis direction as illustrated in FIG. 4. The scanning table 312 may include a bed board and a support structure. The bed board may extend along the longitudinal direction, i.e., the Z-axis direction as illustrated in FIG. 4. The support structure may be configured to support the bed board. In some embodiments, the scanning table 312 may be moved by moving the support structure. In some embodiments, the gantry 410 and the scanning table 312 may be connected by a stretchable plate. Alternatively, the gantry 410 and the scanning table 312 may be detached.

In some embodiments, the object may lie on the scanning table 312 along the Z-axis direction during a scan process. The object that is on the scanning table 312 may be pushed into the inspection chamber along the Z-axis direction. The Z-axis direction may be a moving direction of the scanning table 312. The radiation source 420 may rotate around the Z-axis and emit a beam of X-rays. The detector 430 may be disposed opposite to the radiation source 420 and rotate with the radiation source 420 synchronously. The detector 430 may collect scanning data during the rotation. The scanning data may be raw data received by the detector 430 after the X-rays passed the object, which may be used for imaging. In some embodiments, the detector 430 may transmit the collected scanning data to the host computer 314 after the scan process. The host computer 314 may reconstruct one or more images of the object according to the collected scanning data, and assist a user (e.g., a doctor) to diagnose the object. The host computer 314 may also generate a control signal by analyzing the collected scanning data. The host computer 314 may further transmit the control signal to the mobile device 313 to adjust the movement of the scanning device 311 and/or scanning table 312.

In some embodiments, the scanning table 312 may move with a certain velocity in the inspection chamber. The moving velocity of the scanning table 312 may be adjusted based on different conditions of the object. The moving velocity may include a moving velocity of the scanning table 312 along the Z-axis direction (also referred to as a moving velocity along the axial extension), a moving velocity of the scanning table 312 along the X-axis direction (also referred to as a moving velocity along the radial extension), a moving velocity along the Y-axis direction (also referred to as a moving velocity relative to the horizontal plane), a moving velocity set by a user, or the like, or any combination thereof. In some embodiments, the scanning table 312 may move along the Z-axis direction with an accelerated velocity, a decelerated velocity, or a uniform velocity.

In some embodiments, according to different conditions of the object, the positions of the scanning table 312 and/or the gantry 410 may be adjusted to scan different body parts of the object. The positions of the scanning table 312 and/or the gantry 410 may include the starting positions and/or the end positions of the scanning table 312 and/or the gantry 410. The positions of scanning table 312 and/or the gantry 410 may refer to positions of the scanning table 312 and/or the gantry 410 with respect to the medical imaging system 310. The position(s) may include a position of the scanning table 312 along the Z-axis direction (also referred to as a position along the axial extension), a position along the X-axis direction (also referred to as a position along the radial extension), a position along the Y-axis direction (also referred to as a position relative to the horizontal plane), a tilt angle of the gantry 410 relative to the horizontal plane, or the like, or any combination thereof. In some embodiments, the positions of the scanning table 312 and/or the gantry 410 may be different when different body parts of the object are scanned. For example, the scanning table 312 and/or the gantry 410 may have different positions when a head of the object and a leg of the object are scanned respectively. When the head of the object and the leg of the object are scanned respectively, the position of the scanning table 312 along the Z-axis direction may be different, and the position of the gantry 410 may be the same or different according to scan requirements.

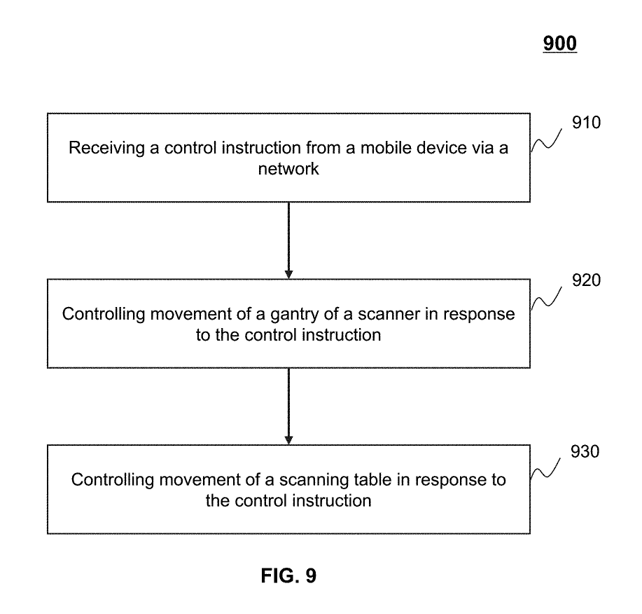

In some embodiments, before a scan process, the gantry 410 and/or the scanning table 312 may be moved to an initial state. The initial state may include a state and/or position of the gantry 410, a state and/or position of the scanning table 312, a position relationship between the gantry 410 and the scanning table 312 before the scan process. In some embodiments, during a scan process, the gantry 410 and/or the scanning table 312 may be moved to finish the scan process. More descriptions of the movement of the gantry and/or the scanning table may be found elsewhere in the present disclosure (e.g., FIG. 9 and the descriptions thereof).

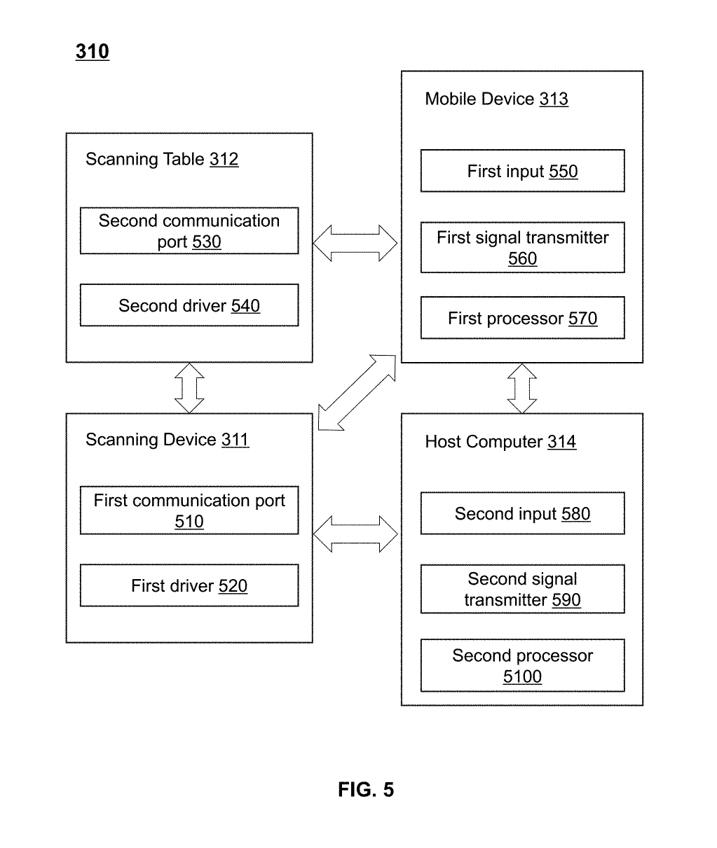

FIG. 5 is a schematic diagram illustrating an exemplary medical imaging system 310 according to some embodiments of the present disclosure. The medical imaging system 310 may include a scanning device 311, a scanning table 312, a mobile device 313, and a host computer 314 (also referred to as a processing device). The mobile device 313 may be used to emit control signals to control the movement of the scanning device 311 and/or the scanning table 312. The host computer 314 may be used to transmit the control signals to the mobile device 313. The gantry 410 and the scanning table 312 may be moved based on the received control signals from the mobile device 313.

The mobile device 313 may include a first input 550, a first signal transmitter 560, and a first processor 570. In some embodiments, the mobile device 313 may also include a display. The first input 550 may be configured to input a first signal. In some embodiments, the first input 550 may receive a control signal (also referred to as “a first signal”) inputted by the user through the mobile device 313, and transmit the control signal to the first processor 570. The first signal may include but is not limited to a text signal, an audio signal, a video signal, an image signal, a graphic signal, or the like, or any combination thereof. The first input 550 may include a keyboard, a touchscreen, a microphone, or the like, or any combination thereof. For example, the user may enter the first signal via a key on the mobile device 313. As another example, the user may enter the first signal via a microphone of the mobile device 313. As a further example, the user may input a graphic as the first signal via a touchscreen of the mobile device 313. After receiving the first signal inputted by the user, the first input 550 may process the signal first and transmit the processed first signal to the first processor 570. Alternatively, the first input 550 may directly transmit the received first signal to the first processor 570. The processing of the first signal may refer to converting the received first signal (e.g., a text signal, an audio signal, a video signal, an image signal, a graphic signal) into an electrical signal (e.g., a digital signal, an analog signal). In some embodiments, the first input 550 may also receive a control signal (also referred to as “a first signal”) from the host computer 314. The control signal may include a digital signal, an analog signal, etc. After receiving the control signal, the first input 550 may directly transmit the control signal to the first signal transmitter 560. Alternatively, after receiving the control signal, the first input 550 may transmit the control signal to the first processor 570. In some embodiments, the first input 550 may also receive a control signal (also referred to as “a first signal”) from the network 330. The control signal may include a digital signal, an analog signal, etc.

In some embodiments, the first signal transmitter 560 may be configured to transmit the processed first signal by the first processor 570 (also referred to as “a second signal”) to the scanning device 311 (e.g., the gantry 410 of the scanning device 311) and/or the scanning table 312. The second signal may include a digital signal, an analog signal, or the like, or any combination thereof.

In some embodiments, the first processor 570 may be configured to process the first signal received from the first input 550 to determine a second signal. The first processor 570 may also be configured to transmit the second signal to the first signal transmitter 560.

The host computer 314 may include a second input 580, a second signal transmitter 590, and a second processor 5100. In some embodiments, the second input 580 may be configured to receive a control signal inputted by a user through the host computer 314, and transmit the control signal to the second processor 5100. The control signal may include but is not limited to a text signal, an audio signal, a video signal, an image signal, a graphic signal, or the like, or any combination thereof. The second input 580 may include a keyboard, a touchscreen, a microphone, or the like, or any combination thereof. For example, a user may input a control signal via a key on the host computer 314. As another example, the user may input a control signal via a microphone of the host computer 314. As a further example, the user may input a graphic as a control signal via a touchscreen of the host computer 314. After receiving the control signal inputted by the user, the second input 580 may process the control signal and transmit the control signal to the second processor 5100. Alternatively, the second input 580 may directly transmit the control signal to the second processor 5100. The processing of the control signal may refer to converting the received control signal (e.g., a text signal, an audio signal, a video signal, an image signal, a graphic signal) into an electrical signal (e.g., a digital signal, an analog signal). In some embodiments, the second input 580 may receive a control signal from the network 330. The control signal may be a digital signal, an analog signal, etc.

In some embodiments, the second input 580 may also obtain scanning data (e.g., raw data) from the scanning device 311. The scanning data may be related to the object. The second input 580 may transmit the scanning data to the second processor 5100.

In some embodiments, the second signal transmitter 590 may obtain the processed control signal from the second processor 5100 and transmit the processed control signal to the mobile device 313. The processed control signal may be a digital signal, an analog signal, or the like, or any combination thereof.

In some embodiments, the second processor 5100 may be configured to process the control signal from the second input 580 and transmit the processed control signal to the second signal transmitter 590. In some embodiments, the second processor 5100 may also be configured to process raw data (or scanning data) from the second input 580.

The scanning device 311 may include a first communication port 510 and a first driver 520. The first communication port 510 may be configured to receive the second signal from the mobile device 313 and transmit the second signal to the first driver 520. The first communication device 510 may also be configured to transmit a feedback from the first driver 520 to the mobile device 313. The feedback may include a real-time position of the gantry 410, or any other information that reflects the status of the scanning device 311. The first driver 520 may be configured to process the second signal from the first communication port 510 and control the movement of the gantry 410 based on the second signal. The first communication port 510 may be mounted on the outer housing of the scanning device 311.

The scanning table 312 may include a second communication port 530 and a second driver 540. The second communication port 530 may be configured to receive the second signal from the mobile device 313 and transmit the second signal to the second driver 540. The second communication port 530 may also be configured to transmit a feedback from the second driver 540 to the mobile device 313. The feedback may include a real-time position of the scanning table 312, a real-time moving velocity of the scanning table 312, or any other information that reflects the status of the scanning table 312. The second driver 540 may be configured to process the second signal from the second communication port 530 and control the movement of the scanning table 312 based on the second signal. The second communication port 530 may be mounted on the support structure of the scanning table 312.

In some embodiments, the first communication port 510 and the second communication port 530 may communicate with each other to implement a self-feedback adjustment between the scanning device 311 and the scanning table 312. The self-feedback adjustment may include adjusting the gantry 410 and/or the scanning table 312 to target positions, or the like.

It should be noted that the above description is merely provided for illustration purposes, and is not intended to limit the protection scope of the present disclosure. In some embodiments, one component may be split into multiple components. Alternatively, multiple components may be integrated into one component. For example, the first input 550, the first signal transmitter 560, and the first processor 570 of the mobile device 313 may be integrated into one component. However, those variations and modifications do not depart from the scope of the present utility. For persons having ordinary skills in the art, multiple variations and modifications may be made under the teachings of the present disclosure. However, those variations and modifications do not depart from the scope of the present disclosure.

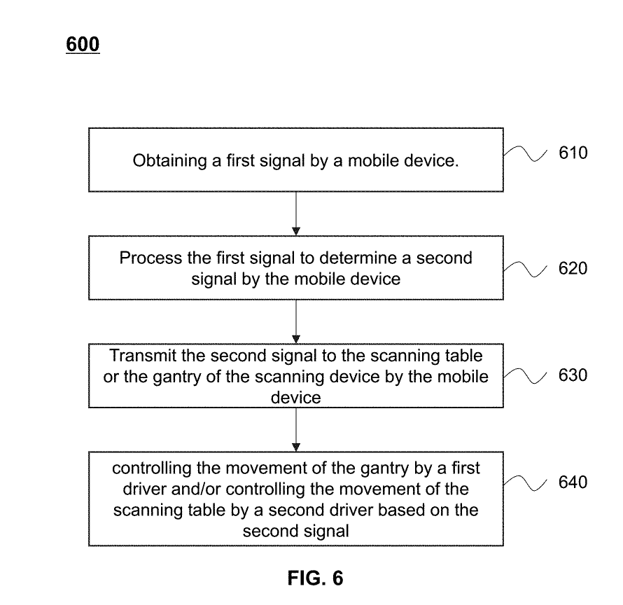

FIG. 6 is a flowchart illustrating an exemplary process 600 for controlling movement of a gantry and/or a scanning table according to some embodiments of the present disclosure.

In 610, the mobile device 313 may obtain a first signal. The mobile device 313 may obtain the first signal inputted by a user via the first input 550 of the mobile device 313. Alternatively, the mobile device 313 may obtain the first signal from the network 330 and/or the host computer 314 via the first input 550 of the mobile device 313. If the first signal is a signal obtained from the network 330 and/or the host computer 314, the first signal may include a digital signal, an analog signal, or the like, or any combination thereof. If the first signal is a signal inputted by the user via the mobile device 313, the first signal may include a text signal, an audio signal, a video signal, an image signal, a graphic signal, or the like, or any combination thereof. In some embodiments, after an object lies on the scanning table 312, the mobile device 313 may start to receive the first signal and the host computer 314 may start to receive the scanning data.

In 620, the mobile device 313 may process the first signal to determine a second signal. The first processor 570 of the mobile device 313 may process the first signal to determine the second signal. The second signal may be a signal that can be recognized by the first driver 520 and/or second driver 540. The second signal may be a digital signal, an analog signal, or the like, or any combination thereof.

In 630, the mobile device 313 may transmit the second signal to the scanning device 311 (e.g., the gantry 410 of the scanning device 311) and/or the scanning table 312. The second signal may be transmitted via a wireless connection, for example, a Bluetooth connection, a WLAN connection, a Wi-Fi connection, or the like, or any combination thereof. The first communication port 510 may receive the second signal from the mobile device 313. The second communication port 530 may receive the second signal from the mobile device 313.

In 640, the first driver 520 may control the movement of the gantry 410 and/or the second driver 540 may control the movement of the scanning table 312 based on the second signal. The first communication port 510 may transmit the received second signal to the first driver 520. The first driver 520 may control the position of the gantry 410 based on the second signal. The position of the gantry 410 may include a tilt angle of the gantry 410 on a XY plane shown in FIG. 4, or the like. The second communication port 530 may transmit the received second signal to the second driver 540. The second driver 540 may control the movement and/or the position of the scanning table 312 based on the second signal. The position of the scanning table 312 may include a position of the scanning table 312 along the Z-axis direction, a position of the scanning table 312 along the X-axis direction, a position of the scanning table 312 along the Y-axis direction, or the like. The movement of the scanning table 312 may include a moving velocity of the scanning table 312 along the Z-axis direction, a moving velocity of the scanning table 312 along the X-axis direction, a moving velocity of the scanning table 312 along the Y-axis direction, a moving velocity along other directions, or the like. It should be noted that the positions of the scanning table 312 and/or the gantry 410 may refer to positions of the scanning table 312 and/or the gantry 410 relative to medical imaging system 310.

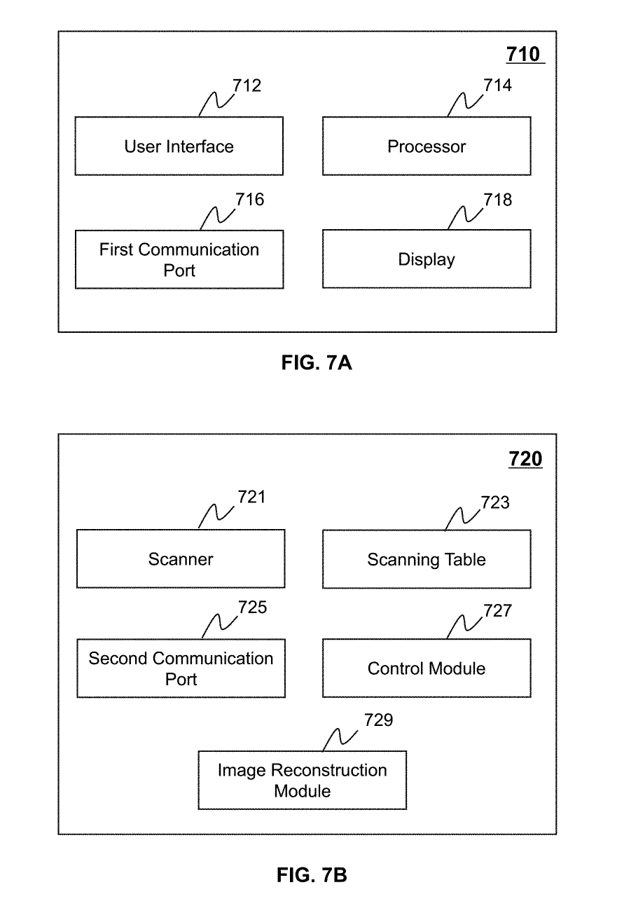

FIG. 7A is a block diagram illustrating an exemplary mobile device 710 according to some embodiments of the present disclosure. As illustrated, the mobile device 710 may include a user interface 712, a processor 714, a first communication port 716, and a display 718.

The user interface 712 may be configured to support data and/or information interactions between the mobile device 710 and a user. In some embodiments, the mobile device 710 may receive information entered by a user via the user interface 712. The information received may include data related to an object, one or more scanning parameters, one or more reconstruction parameters, or the like, or any combination thereof. Merely by way of example, the data related to the object may include basic information, medical information, diagnostic information, or the like or any combination thereof. The basic information may include name, gender, age, height, weight, profession, marriage, native place, workplace, home address, etc. The medical information may include current disease condition, medical history, family medical history, medicine allergy, infectious diseases, etc. The diagnostic information may include diagnostic result, therapeutic regimen, assay report, etc. Merely by way of example, the scanning parameter(s) may include scanning time, irradiation dose, slice thickness of scanning, tube voltage, tube current, tilt angle of a gantry, height of the gantry, rotation velocity of the gantry, tilt angle of a scanning table, moving velocity of the scanning table, or the like, or any combination thereof. In some embodiments, part of the scanning parameter(s) may be default settings of a scanner. Part of the scanning parameter(s) may be determined based on empirical value, or by analyzing data related to an object, for example, based on a scan plan. Merely by way of example, the reconstruction parameter(s) may include a reconstruction function, a size of a reconstructed image, iteration times of reconstruction, a size of a reconstruction matrix, slice thickness of reconstruction, or the like, or any combination thereof. In some embodiments, part of the reconstruction parameter(s) may be default settings of the scanner. Part of the reconstruction parameter(s) may be determined based on empirical value, or by analyzing data related to an object, or the scanning data.

In some embodiments, the information received may include text information, audio information, video information, image information, graphic information, or the like, or any combination thereof. In some embodiments, the user interface 712 may include an input and/or an output (I/O). The information may be received by the mobile device 710 via the input, including a character input device (e.g., a keyboard), an optical reading device (e.g., an optical mark reader, an optical character reader, etc.), a graphic input device (e.g., a mouse, a joystick, a light pen, etc.), an image input device (e.g., a camera, a scanner, a fax machine, etc.), an analog input device (e.g., a language analog-digital conversion recognition system), or the like, or any combination thereof. In some embodiments, the mobile device 710 may obtain the information from the network 120.

The processor 714 may be configured to generate an instruction based on one or more user inputs, for example, based on the information received from the user interface 712. In some embodiments, the processor 714 may generate a scan instruction based on the data related to an object, the scanning parameter(s), etc. For example, the received information may include one or more scanning parameters, such as scanning time, irradiation dose. The processor 714 may generate a scan instruction based on the scanning parameter(s) received. As another example, the inputted information may include data related to an object, such as the medical information, the diagnostic information. The processor 714 may analyze the data related to the object to generate one or more corresponding scanning parameters. The processor 714 may also generate a scan instruction based on the corresponding scanning parameter(s). In some embodiments, the scan instruction may be used to adjust one or more parameters of a scanner (e.g., the scanner 721). The scan instruction may also cause the scanner (e.g., the scanner 721) to scan an object and generate scanning data related to the object.

In some embodiments, the processor 714 may generate a control instruction based on the data related to an object, the scanning parameter(s), etc. For example, the received information may include one or more scanning parameters, such as a tilt angle of a gantry, rotation velocity of the gantry, a tilt angle of a scanning table. The processor 714 may generate a control instruction based on the scanning parameter(s) received. As another example, the received information may include data related to the object, such as age, weight, height. The processor 714 may analyze the data related to the object to generate one or more corresponding scanning parameters. The processor 714 may also generate a control instruction based on the corresponding scanning parameter(s). The control instruction may be used to control the movement of a gantry of the scanner 721 or the scanning table 723.

In some embodiments, the processor 714 may generate a data processing instruction based on the reconstruction parameters. For example, the inputted information may include one or more reconstruction parameters, such as a reconstruction function, a size of a reconstructed image. The processor 714 may generate a data processing instruction based on the reconstruction parameter(s) received. In some embodiments, the data processing instruction may be used to cause a data processing module to manage and/or process data, including data related to the object, image data, file data, training data, or the like, or any combination thereof. For example, the data processing instruction may be used to manage and/or process data related to the object, including addition, deletion, ranking, screening, analyzing, registration, etc. In some embodiments, the data processing instruction may also be used to manage and/or process images, including image reconstruction, image segmentation, image amplification, image reduction, image processing, or the like, or any combination thereof. For example, the data processing instruction may cause an image reconstruction module (e.g., an image reconstruction module 729) to reconstruct one or more images of an object based on the scanning data generated by the scanner 721. The image(s) may include two-dimensional (2D) image(s), three-dimensional (3D) image(s), four-dimensional (4D) image(s), or the like, or any combination thereof.

The first communication port 716 may be connected to a network (e.g., the network 120) and be configured to enable communications between the mobile device 710 and the medical imaging device 720. The network 120 may include a local area network (LAN), a wide area network (WAN), a wireless local area network (WLAN), a metropolitan area network (MAN), a WI-FI network, a cellular communication network (e.g., GSM, CDMA, 2G, 3G, 4G, 5G, etc.), a short-range radio communication network (e.g., Bluetooth, ZigBee, near field communication (NFC), radio frequency identification (RFID)), or the like, or any combination thereof. In some embodiments, the first communication port 716 may transmit an instruction to the medical imaging device 720 via the network 120, causing the medical imaging device 720 to operate in response to the instruction. The instruction may be transmitted to the medical imaging device 720 in the form of digital signals and/or analog signals. For example, the first communication port 716 may transmit a scan instruction to the second communication port 725 of the medical imaging device 720 via the network 120. The scan instruction may cause the scanner 721 of the medical imaging device 720 to scan an object and generate scanning data related to the object. As another example, the first communication port 716 may transmit a data processing instruction to the second communication port 725 of the medical imaging device 720 via the network 120. The data processing instruction may cause the image reconstruction module 729 of the medical imaging device 720 to reconstruct one or more images of the object based on the scanning data related to the object. In some embodiments, the first communication port 716 may receive information from the medical imaging device 720 via the network 120. For example, the first communication port 716 may receive the scanning data related to the object from the medical imaging device 720 via the network 120. As another example, the first communication port 716 may receive the image(s) of the object from the medical imaging device 720 via the network 120.

The display 718 may include a screen. In some embodiments, the display 718 may be configured to present information entered by a user via the user interface 712. For example, the display may present data related to the object (e.g., basic information, medical information, diagnostic information). In some embodiments, the display 718 may be used to present information received wirelessly from the medical imaging device 720. For example, the display 718 may present the scanning data wirelessly received from the medical imaging device 720. As another example, the display 718 may present the image(s) received wirelessly from the medical imaging device 720.

It should be noted that the above description of the mobile device 710 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. For example, the mobile device 710 may further include a storage. The information entered via the user interface 712, scanning data and/or one or more images received from the medical imaging device 720 via the network 120 by the first communication port 716 may be stored in the storage.

FIG. 7B is a block diagram illustrating an exemplary medical imaging device 720 according to some embodiments of the present disclosure. As illustrated, the medical imaging device 720 may include a scanner 721, a scanning table 723, a second communication port 725, a control module 727, and an image reconstruction module 729.

The scanner 721 may include a gantry, a detector mounted to the gantry, a radiation source mounted to the gantry opposite the detector, and/or a detection region with a field of view (FOV). In some embodiments, for positron emission tomography (PET), the radiation source may be omitted. Details regarding the detector and the radiation source may be found elsewhere in the present disclosure (e.g., FIG. 1 and the relevant descriptions thereof). In some embodiments, the scanner 721 may scan an object and generate scanning data related to the object in response to a scan instruction. The scan instruction may be received from the mobile device 710 via the network 120 by the second communication port 725. The scan instruction may correspond to one or more scanning parameters, including scanning time, irradiation dose, slice thickness of scanning, tube voltage, tube current, or the like, or any combination thereof. In some embodiments, one or more scanning parameters of the scanner 721 (e.g., tube voltage, tube current of the radiation source, scanning time) may be adjusted based on the scan instruction. Alternatively, the scanner 721 may scan the object and generate scanning data based on the scan instruction.

The gantry of the scanner 721 may include an inspection chamber surrounded by an outer housing. The inspection chamber may have a circular shape and extend along the longitudinal direction (e.g., the Z-axis direction in FIG. 1). In some embodiments, the outer housing of the gantry may include a load-bearing component, such as a groove, a shelf, a fastening structure, etc. The load-bearing component (e.g., the groove, the shelf, the fastening structure) may be used to accommodate the mobile device 710. For example, the outer housing may include a groove, and the mobile device 710 may be put into the groove. As another example, the outer housing may include a shelf, and the mobile device 710 may be put on the shelf. In some embodiments, the outer housing of the gantry may include a charging connector and/or a universal serial bus (USB) port. The charging connector and/or the USB port may be located around or inside the load-bearing component. The mobile device 710 may be charged via the charging connector and/or the USB port. For example, the outer housing may include a charging connector, and the mobile device 710 may include a charging plug matching the charging connector. The charging plug may be inserted into the charging connector, and the mobile device 710 may be charged. As another example, the outer housing may include a first USB port, and the mobile device 710 may include a second USB port. The first USB port and the second USB port may be connected via a USB cable, and the mobile device 710 may be charged. In some embodiments, the mobile device 710 may be charged via a wireless charging technology. The wireless charging technology may include electromagnetic induced charging, resonant charging, etc. In some embodiments, during a scan process, the gantry may rotate with a rotation velocity. The rotation velocity may be controlled by the control module 727 based on a control instruction.

The scanning table 723 may include a bed board and a support structure. The bed board may extend along the longitudinal direction (e.g., the Z-axis direction in FIG. 1). The bed board may be used to accommodate an object. The object may be placed on the scanning table 723 along the Z-axis direction in FIG. 1. The object may include a patient, an animal, a man-made object, etc. The support structure may be used to support the bed board. In some embodiments, the scanning table 723 may be part of the scanner 721. During a scan process, the scanning table 723 may be moved along the z-axis direction into the inspection chamber of the gantry. The movement of the scanning table 723 may be controlled by the control module 727 based on a control instruction.

The second communication port 725 may be configured to receive an instruction from the mobile device 710 via a network (e.g., the network 120). For example, the second communication port 725 may receive a scan instruction from the mobile device 710 via the network 120. As another example, the second communication port 725 may receive a control instruction from the mobile device 710 via the network 120. As a further example, the second communication port 725 may receive a data processing instruction from the mobile device 710 via the network 120. In some embodiments, the second communication port 725 may be further configured to transmit information (e.g., scanning data, one or more images) to the mobile device 710 via the network 120.

The control module 727 may be configured to control the movement of the gantry of the scanner 721 or the scanning table 723 in response to a control instruction. In some embodiments, the control module 727 may include a driver. The driver may generate mechanical energy based on the control instruction. Then the driver may control the movement of the gantry of the scanner 721, the scanning table 723, etc. based on the control instruction. Details regarding the movement of the gantry of the scanner 721 or the scanning table 723 may be found elsewhere in the present disclosure (e.g., FIG. 9 and the relevant description thereof).

The image reconstruction module 729 may be configured to reconstruct one or more images based on scanning data in response to a data processing instruction. The image(s) may include two-dimensional (2D) image(s), three-dimensional (3D) image(s), four-dimensional (4D) image(s), or the like, or any combination thereof. The data processing instruction may be received from the mobile device 710 via the network 120 by the second communication port 725. The data processing instruction may correspond to one or more reconstruction parameters, including a reconstruction function, a size of a reconstructed image, iteration times of reconstruction, a size of a reconstruction matrix, slice thickness of reconstruction, or the like, or any combination thereof. In some embodiments, the image reconstruction module 729 may be further configured to process the image(s) in response to a data processing instruction. The processing of the image(s) may include image segmentation, image amplification, image reduction, image processing, or the like, or any combination thereof.

It should be noted that the above description of the medical imaging device 720 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. For example, the medical imaging device 720 may further include a storage module. The scanning data generated by the scanner 721 and/or one or more images generated by the image reconstruction module 729 may be stored in the storage module. In some embodiments, the mobile device 710 may be part of the medical imaging device 720.

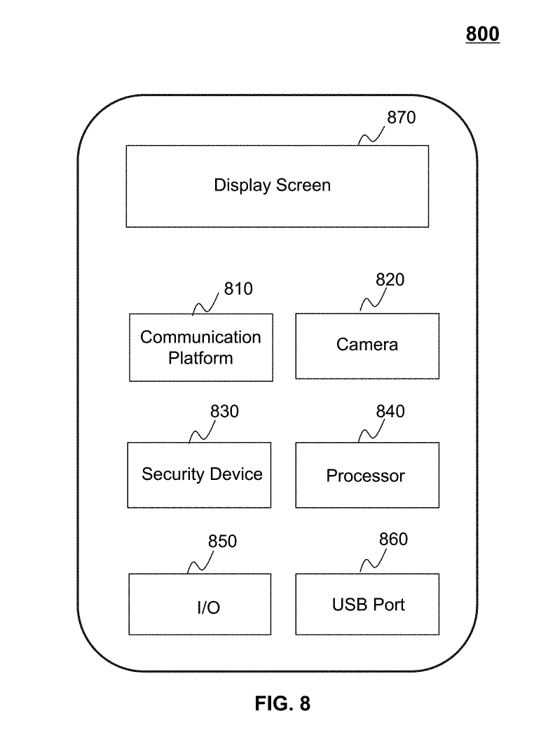

FIG. 8 is a schematic diagram illustrating an exemplary mobile device 800 according to some embodiments of the present disclosure. In some embodiments, the mobile device 800 may be a tablet PC (e.g., an iPad™). As illustrated, the mobile device 800 may include a communication platform 810, a camera 820, a security device 830, a processor 840, an I/O 850, a USB port 860, and a display screen 870.

The communication platform 810 may include a first communication port (e.g., the first communication port 716) enable communications between the mobile device 800 and a medical imaging device (e.g., the medical imaging device 720) via the network 120. The camera 820 may be used to take photos or scan identification codes. The identification codes may include Quick Response (QR) codes, barcodes, etc. For example, before scanning, a user (e.g., a doctor) may take photos of an object (e.g., a patient) using the camera 820 to record data related to the object (e.g., the patient), for example, a size and/or a shape of a wound related to the object. As another example, before scanning, a user (e.g., a doctor) may scan a case of an object (e.g., a patient), or an identification code (e.g., a QR code, or a barcode) related to the object (e.g., the patient) using the camera 820 to obtain data related to the object (e.g., the patient). The security device 830 may be used to lock the mobile device 800 to ensure the safety of data. In some embodiments, the security device 830 may include a screen locker, a password locker, or the like, or any combination thereof. The password locker may include gesture password, fingerprint password, digital password, or the like, or any combination thereof. For example, a user (e.g., a doctor) may lock the mobile device 800 by the digital password, and the mobile device 800 can be opened unless the digital password is correctly inputted. The processor 840 may generate an instruction (e.g., a san instruction, a control instruction, a data processing instruction) based on the received information. The information received may include data related to an object, one or more scanning parameters, one or more reconstruction parameters, or the like, or any combination thereof. The I/O 850 may support data and/or information interactions between the mobile device 800 and a user. For example, a user may enter information into the mobile device 800 via the I/O 850. The USB port 860 may be connected to a USB port of the gantry of the scanner 221 and charge the mobile device 800. The display screen 870 may be used to present data and/or image(s). For example, the display screen 870 may present information entered by a user. As another example, the display screen 870 may present scanning data received (wired or wireless) from the medical imaging device 720. As a further example, the display screen 870 may present one or more images received (wired or wireless) from the medical imaging device 720. In some embodiments, the data and/or image(s) may be presented in the form of text, audio, video, picture, or the like, or any combination thereof. Thus, the mobile device 800 may include a loudspeaker (not shown) to display the audio data, the video data, etc.

It should be noted that the above description of the mobile device 800 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. For example, any other suitable component, including but not limited to storage (not shown), may also be included in the mobile device 800. The information inputted via the I/O 850, scanning data and/or one or more images received from the medical imaging device 720 via the network 120 may be stored in the storage.

FIG. 9 is a flowchart illustrating an exemplary process 900 for controlling the movement of a gantry or a scanning table according to some embodiments of the present disclosure. The process 900 may be executed by the medical imaging system 100. For example, the process 900 may be implemented as a set of instructions (e.g., an application) stored in a processor of the medical imaging system 100 (e.g., a processor in the processing device 140). The processor in the processing device 140 may execute the set of instructions and may accordingly be directed to perform the process 900. The operations of the illustrated process presented below are intended to be illustrative. In some embodiments, the process 900 may be accomplished with one or more additional operations not described, and/or without one or more of the operations discussed. Additionally, the order in which the operations of the process as illustrated in FIG. 9 and described herein is not intended to be limiting.

In 910, a control instruction may be received from a mobile device (e.g., the mobile device 710) via a network (e.g., the network 120). Merely by way of example, the control instruction may relate to controlling the movement of a gantry (e.g., the gantry of the scanner 721), and/or controlling the movement of a scanning table (e.g., the scanning table 723). In some embodiments, the control instruction may be generated by the processor 714 of the mobile device 710 based on data related to an object, one or more scanning parameters, etc. For example, the processor 714 may generate a control instruction based on the scanning parameter(s), such as tilt angle of a gantry, rotation velocity of the gantry, tilt angle of a scanning table, etc. The control instruction may be generated before or during a scan process. In some embodiments, the control instruction may be in the form of a digital signal and/or analog signal. A medical imaging device (e.g., the second communication port 725 of the medical imaging device 720) may receive the control instruction (e.g., the digital signal and/or analog signal) via the network (e.g., the network 120).

In 920, the movement of a gantry of a scanner (e.g., the gantry of the scanner 721) may be controlled in response to the control instruction. In some embodiments, the control instruction may relate to controlling the movement of the gantry. The medical imaging device (e.g., the control module 727 of the medical imaging device 720) may control the movement of the gantry of the scanner 721 in response to the control instruction. Specifically, the control module 727 may include a driver. The driver may generate mechanical energy to move the gantry based on the control instruction. In some embodiments, the control module 727 may perform at least one of: adjusting a tilt angle of the gantry relative to a horizontal plane, controlling a deflection angle of the gantry, controlling a rotation velocity of the gantry, and/or controlling a rotation direction of the gantry. The tilt angle of the gantry relative to the horizontal plane may relate to a tilt angle of a rotation axis of the gantry (i.e., the Z-axis direction in FIG. 1) relative to the horizontal plane, and in general, the tilt angle may be set as zero. The deflection angle of the gantry may relate to an initial angle of the gantry. The initial angle of the gantry may relate to a line connecting the radiation source and the detector, which are oppositely mounted on the gantry. If the line connecting the radiation source and the detector is vertical, the initial angle may be designated as zero. The rotation velocity of the gantry may relate to a velocity of the gantry rotating along the rotation axis. In some embodiments, the rotation velocity of the gantry may be determined based on a scan plan, such as the scanning time, the radiation dose, the scan region of the object, etc. The rotation direction of the gantry may include a counterclockwise rotation or a clockwise rotation.

In some embodiments, the control module 727 may control the movement of the gantry of the scanner 721 before the scan process. For example, before the scan process, the control module 727 may adjust the tilt angle of the gantry to be zero (e.g., the rotation axis of the gantry being parallel to the horizontal plane) in response to the control instruction. As another example, before the scan process, the control module 727 may adjust a deflection angle of the gantry (e.g., an initial angle of the gantry) to be zero in response to the control instruction. In some embodiments, the control module 727 may control the movement of the gantry during a scan process. For example, the control module 727 may adjust the rotation velocity or the rotation direction of the gantry during the scan process in response to the control instruction.

In 930, the movement of a scanning table (e.g., the scanning table 723) may be controlled in response to the control instruction. In some embodiments, the control instruction may be related to controlling the movement of the scanning table (e.g., the scanning table 723). The medical imaging device (e.g., the control module 727 of the medical imaging device 720) may control the movement of the scanning table (e.g., the scanning table 723) in response to the control instruction. The driver of the control module 727 may generate mechanical energy to move the scanning table 723 based on the control instruction. In some embodiments, the control module 727 may perform at least one of controlling a moving distance of the scanning table 723 with respect to the gantry of the scanner 721, controlling a moving direction of the scanning table 723 with respect to the gantry, controlling a moving velocity of the scanning table 723, controlling a moving acceleration of the scanning table 723, and/or controlling a tilt angle of the scanning table 723. The moving distance and/or moving direction of the scanning table 723 may relate to an actual positional relationship between the gantry of the scanner 721 and the scanning table 723, and the desired positional relationship thereof. In some embodiments, the positional relationship (e.g., the actual positional relationship, the desired positional relationship) may indicate a distance between the gantry of the scanner 721 and the scanning table 723 (e.g., a distance between a head of the scanning table 723 and a front end of the gantry). In some embodiments, the moving velocity of the scanning table 723 may include a uniform velocity, a non-uniform velocity, etc. The moving acceleration of the scanning table 723 may indicate a change in the moving velocity of the scanning table 723 divided by time. In some embodiments, the moving acceleration of the scanning table 723 may include a uniform acceleration, a non-uniform acceleration, a positive acceleration, a negative acceleration, etc. The tile angle of the scanning table 723 may relate to an angle of the bed board of the scanning table 723 relative to the horizontal plane.

In some embodiments, the control module 727 may control the movement of the scanning table 723 before the scan process. For example, before the scan process, the control module 727 may move the scanning table 723 towards or away from the gantry of the scanner 721 in response to the control instruction. As another example, before the scan process, the control module 727 may adjust the tile angle of the scanning table to be zero. In some embodiments, the control module 727 may control the movement of the scanning table 723 during the scan process. For example, the control module 727 may control the moving distance and/or moving direction of the scanning table 723 in the inspection chamber of the gantry during the scan process. If an object (e.g., a patient) on the scanning table 723 has finished a head scan and is to start a chest scan, the control module 227 may control the moving distance and/or moving direction of the scanning table 312 in the inspection chamber of the gantry 410. As another example, the control module 727 may control the moving velocity and/or moving acceleration of the scanning table 723 during the scan process. In some embodiments, the moving velocity and/or the moving acceleration may be default settings of the medical imaging device or adjusted before or during the scan process by a user (e.g., a doctor).

It should be noted that the above description of the process 900 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, the control module 727 may perform steps 920 and 930 in any order. For example, the control module 727 may perform steps 920 and 930 simultaneously. As another example, the control module 727 may perform step 920 before or after step 930.

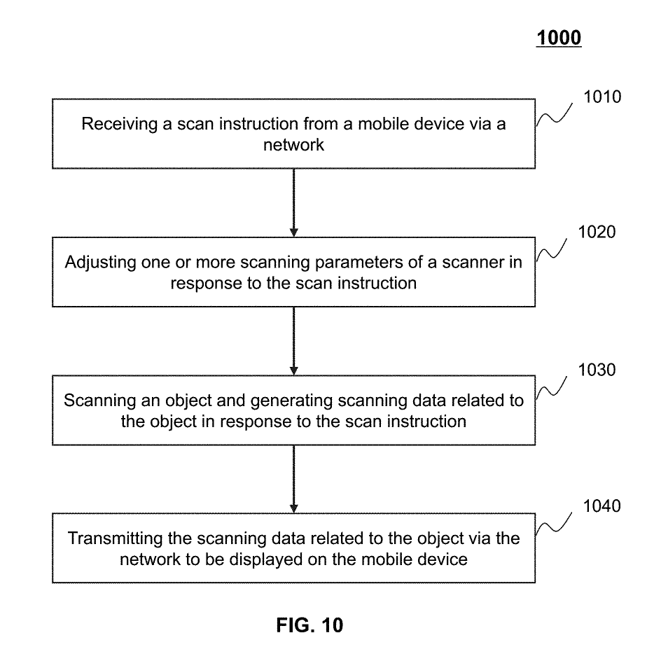

FIG. 10 is a flowchart illustrating an exemplary process 1000 for scanning an object according to some embodiments of the present disclosure. The process 1000 may be executed by the medical imaging system 100. For example, the process 1000 may be implemented as a set of instructions (e.g., an application) stored in a processor of the medical imaging system 100 (e.g., a processor in the processing device 140 illustrated in FIG. 1). The processor in the processing device 140 may execute the set of instructions and may accordingly be directed to perform the process 1000. The operations of the illustrated process presented below are intended to be illustrative. In some embodiments, the process 1000 may be accomplished with one or more additional operations not described, and/or without one or more of the operations discussed. Additionally, the order in which the operations of the process as illustrated in FIG. 10 and described herein is not intended to be limiting.

In 1010, a scan instruction may be received from a mobile device (e.g., the mobile device 710) via a network (e.g., the network 120). Merely by way of example, the scan instruction may relate to adjusting one or more scanning parameters of a scanner (e.g., the scanner 721), scanning an object and generating scanning data related to the object, etc. In some embodiments, the scan instruction may be generated by the processor 714 of the mobile device 710 based on data related to an object, one or more scanning parameters, etc. For example, the processor 714 may generate a scan instruction based on the scanning parameter(s) (e.g., scanning time, irradiation dose). In some embodiments, the scan instruction may be in the form of a digital signal and/or analog signal. A medical imaging device (e.g., the second communication port 725 of the medical imaging device 720) may receive the scan instruction via the network (e.g., the network 120).

In 1020, one or more scanning parameters of a scanner (e.g., the scanner 721) may be adjusted in response to the scan instruction. In some embodiments, the scan instruction may relate to adjusting the scanning parameter(s) of the scanner (e.g., the scanner 721). In some embodiments, the scanning parameter(s) may include scanning time, irradiation dose, slice thickness of scanning, tube voltage, tube current, or the like, or any combination thereof. The scanner (e.g., a processor of the scanner 721) may adjust the scanning parameter(s) in response to the scan instruction.

In 1030, an object may be scanned in response to the scan instruction. In some embodiments, the scan instruction may relate to scanning an object and generating scanning data related to the object. In some embodiments, the scanner (e.g., the scanner 721) may perform a scan process to scan the object in response to the scan instruction. The scanner (e.g., the scanner 721) may generate scanning data related to the object during the scan process. In some embodiments, the scanning data related to the object may include a plurality of data for reconstruction.

In 1040, the scanning data related to the object may be transmitted via the network (e.g., the network 120) to be displayed on the mobile device (e.g., the mobile device 710). In some embodiments, the medical imaging device (e.g., the second communication port 725 of the medical imaging device 720) may transmit the scanning data to the mobile device (e.g., the mobile device 710) via the network (e.g., the network 120). The scanning data may be displayed on a display (e.g., the display 718). A user (e.g., a doctor) may look through the scanning data to make a further scan plan or a treatment plan. In some embodiments, the scanning data may be stored in a storage of the medical imaging device for further processing, for example, image reconstruction.

It should be noted that the above description of the process 1000 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. For example, step 1020 may be omitted, and one or more default scanning parameters may be applied during the scan process.

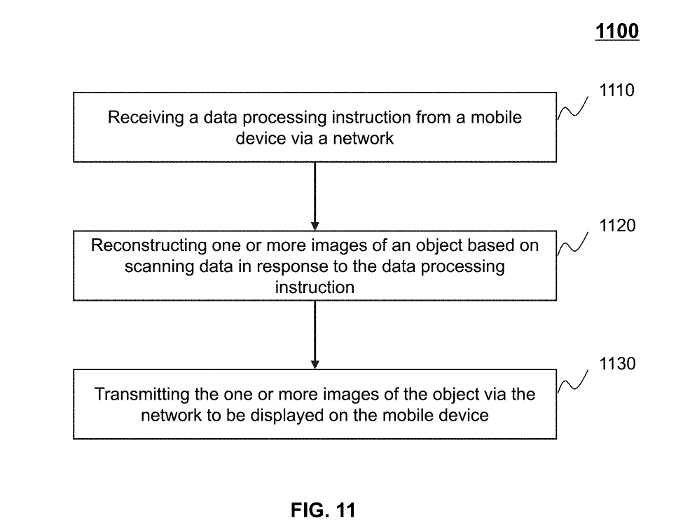

FIG. 11 is a flowchart illustrating an exemplary process 1100 for reconstructing one or more images according to some embodiments of the present disclosure. The process 1100 may be executed by the medical imaging system 100. For example, the process 1100 may be implemented as a set of instructions (e.g., an application) stored in a processor of the medical imaging system 100 (e.g., a processor in the processing device 140). The processor in the processing device 140 may execute the set of instructions and may accordingly be directed to perform the process 1100. The operations of the illustrated process presented below are intended to be illustrative. In some embodiments, the process 1100 may be accomplished with one or more additional operations not described, and/or without one or more of the operations discussed. Additionally, the order in which the operations of the process as illustrated in FIG. 11 and described herein is not intended to be limiting.

In 1110, a data processing instruction may be received from a mobile device (e.g., the mobile device 710) via a network (e.g., the network 120). The data processing instruction may relate to image reconstruction based on scanning data. In some embodiments, the data processing instruction may be generated by the processor 714 of the mobile device 710. For example, the processor 714 may generate the data processing instruction based on one or more reconstruction parameters (e.g., a reconstruction function, iteration times of reconstruction). In some embodiments, the data processing instruction may be in the form of a digital signal and/or analog signal. A medical imaging device (e.g., the second communication port 725 of the medical imaging device 720) may receive the data processing instruction via the network (e.g., the network 120).

In 1120, one or more images of an object may be reconstructed based on scanning data in response to the data processing instruction. In some embodiments, the medical imaging device (e.g., the image reconstruction module 729 of the medical imaging device 720) may reconstruct the image(s) based on the scanning data in response to the data processing instruction. The scanning data may be generated during a scan process (e.g., as described elsewhere in the present disclosure in connection with FIG. 10). In some embodiments, the image(s) may include two-dimensional (2D) image(s), three-dimensional (3D) image(s), four-dimensional (4D) image(s), or the like, or any combination thereof.

In 1130, the one or more images of the object may be transmitted via the network (e.g., the network 120) to be displayed on the mobile device (e.g., the mobile device 710). In some embodiments, the medical imaging device (e.g., the second communication port 725 of the medical imaging device 720) may transmit the image(s) to the mobile device (e.g., the mobile device 710) via the network (e.g., the network 120). The image(s) may be displayed on a display (e.g., the display 718). A user (e.g., a doctor) may look through the image(s) to make a further scan plan or a treatment plan. In some embodiments, the image(s) may be stored in a storage of the medical imaging device.

It should be noted that the above description of the process 1100 is provided for the purposes of illustration, and is not intended to limit the scope of the present disclosure. For persons having ordinary skills in the art, multiple variations and modifications may be made under the teachings of the present disclosure. However, those variations and modifications do not depart from the scope of the present disclosure. In some embodiments, the data processing instruction may relate to other data processing, such as imaging segmentation, image amplification, image reduction, etc. The medical imaging device may further process the reconstructed image(s) based on the data processing instruction.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A non-transitory computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A medical imaging device including at least one processor, at least one storage and a communication platform connected to a network, the medical imaging device comprising:
a scanner configured to generate scanning data related to an object, including
a gantry;
a radiation source mounted to the gantry and configured to emit radiations to the object; and
a detector mounted to the gantry opposite to the radiation source, and configured to detect radiations emitted from the radiation source;
a scanning table configured to move with respect to the gantry of the scanner;
a control module configured to control a movement of at least one of the gantry of the scanner or the scanning table; and
a communication port configured to receive an instruction from a mobile device via the network; wherein
the scanner scans the object and generates the scanning data related to the object in response to a scan instruction received by the communication port from the mobile device via the network; and/or
the control module controls the movement of at least one of the gantry of the scanner or the scanning table in response to a control instruction received by the communication port from the mobile device via the network.

2. The medical imaging device of claim 1, wherein to control the movement of the gantry of the scanner, the control module is further configured to perform at least one of:
adjusting a tilt angle of the gantry relative to a horizontal plane,
controlling a deflection angle of the gantry,
controlling a rotation velocity of the gantry, or
controlling a rotation direction of the gantry.

3. The medical imaging device of claim 1, wherein to control the movement of the scanning table, the control module is further configured to perform at least one of:
controlling a moving distance of the scanning table with respect to the gantry,
controlling a moving direction of the scanning table with respect to the gantry,
controlling a moving velocity of the scanning table,
controlling a moving acceleration of the scanning table, or
controlling a tilt angle of the scanning table.

4. The medical imaging device of claim 1, further comprising an image reconstruction module, wherein
the image reconstruction module is configured to reconstruct one or more images of the object based on the scanning data related to the object in response to a data processing instruction received by the communication port from the mobile device via the network.

5. The medical imaging device of claim 4, wherein the communication port is further configured to transmit at least one of the scanning data related to the object or the one or more images of the object via the network to be displayed on the mobile device.

6. The medical imaging device of claim 1, wherein the gantry of the scanner is configured with at least one of a Universal Serial Bus (USB) port or a charging connector to charge the mobile device.

7. A medical imaging method implemented on one machine including at least one processor, at least one storage, and a communication platform connected to a network, the method comprising:
receiving a scan instruction from a mobile device via the network;
generating scanning data related to an object by scanning the object in response to the scan instruction;
receiving a control instruction form the mobile device via the network; and
controlling movement of at least one of a gantry of a scanner or a scanning table in response to the control instruction.

8. The medical imaging method of claim 7, wherein controlling the movement of the gantry of the scanner, the method comprises performing at least one of:
adjusting a tilt angle of the gantry relative to a horizontal plane,
controlling a deflection angle of the gantry,
controlling a rotation velocity of the gantry, or
controlling a rotation direction of the gantry.

9. The medical imaging method of claim 7, wherein controlling the movement of the scanning table, the method comprises performing at least one of:
controlling a moving distance of the scanning table with respect to the gantry,
controlling a moving direction of the scanning table with respect to the gantry,
controlling a moving velocity of the scanning table,
controlling a moving acceleration of the scanning table, or
controlling a tilt angle of the scanning table.

10. The medical imaging method of claim 7, further comprising:
receiving a data processing instruction from the mobile device via the network; and
reconstruct one or more images of the object based on the scanning data related to the object in response to the data processing instruction.

11. The medical imaging method of claim 10, further comprising:
transmitting at least one of the scanning data related to the object or the one or more images of the object via the network to be displayed on the mobile device.

12. (canceled)

13. A medical imaging system, comprising:
a mobile device configured to remotely control a medical imaging device; and
the medical imaging device including:
a scanner configured to generate scanning data related to an object;
a control module configured to control a movement of at least one of a gantry of the scanner or a scanning table; and
a second communication port configured to receive an instruction from the mobile device via a network, wherein
the scanner scans the object and generates scanning data related to the object in response to a scan instruction received by the second communication port from the mobile device via the network; and
the control module controls the movement of at least one of the gantry of the scanner or the scanning table in response to a control instruction received by the second communication port from the mobile device via the network.

14. The medical imaging system of claim 13, wherein to control the movement of the gantry of the scanner, the control module is further configured to perform at least one of:
adjusting a tilt angle of the gantry relative to a horizontal plane,
controlling a deflection angle of the gantry,
controlling a rotation velocity of the gantry, or
controlling a rotation direction of the gantry.

15. The medical imaging system of claim 13, wherein to control the movement of the scanning table, the control module is further configured to perform at least one of:
controlling a moving distance of the scanning table with respect to the gantry,
controlling a moving direction of the scanning table with respect to the gantry,
controlling a moving velocity of the scanning table,
controlling a moving acceleration of the scanning table, or
controlling a tilt angle of the scanning table.

16. The medical imaging system of claim 13, further comprising an image reconstruction module, wherein
the image reconstruction module is configured to reconstruct one or more images of the object based on the scanning data related to the object in response to a data processing instruction received by the second communication port from the mobile device via the network.

17. The medical imaging system of claim 16, wherein the second communication port is further configured to transmit at least one of the scanning data related to the object or the one or more images of the object via the network to be displayed on the mobile device.

18. The medical imaging system of claim 13, wherein the mobile device comprises:
at least one processor;
a user interface;
a display; and
a first communication port connected to the network and configured to enable communications between the mobile device and the medical imaging device,
wherein
the at least one processor is configured to generate an instruction based on one or more user inputs, and
the first communication port is configured to transmit the instruction to the medical imaging device, causing the medical imaging device to operate in response to the instruction.

19. (canceled)

20. The medical imaging system of claim 18, wherein the mobile device further includes:
a security device configured to lock the mobile device.

21. The medical imaging system of claim 13, wherein the scanner includes at least one of a Universal Serial Bus (USB) port or a charging connector to charge the mobile device.

22. The medical imaging system of claim 21, wherein the scanner includes:
an outer housing, wherein the outer housing includes a load-bearing component, the load-bearing component used to accommodate the mobile device, the USB port or the charging connector being located around or inside the load-bearing component; and
an inspection chamber surrounded by the outer housing and extended along a longitudinal direction, the inspection chamber used to accommodate the scanning table.

23-42. (canceled)
